(19) United States
(12) Patent Application Publication
(10) Pub. No.: US 2012/0191037 A1
(43) Pub. Date: Jul. 26, 2012

(54) TUBE CONNECTOR WITH INTEGRATED RELIEF VALVE

(75) Inventors: Niles Patel, Westlake, OH (US); Jeffrey B. Williams, Hudson, OH (US)

(73) Assignee: Thermedx, LLC

(21) Appl. No.: 13/012,835

(22) Filed: Jan. 25, 2011

(51) Int. Cl. A61M 1/00; F16L 33/16; F16L 55/00

(52) U.S. Cl. 604/30; 138/37; 604/246; 285/95

(57) ABSTRACT

A connector comprised of a body having a fluid passageway extending therethrough, wherein a first fitting is formed at a first end of the body and a second fitting is formed at a second end of the body, an opening formed in the body that is in communication with the fluid passageway, and a pressure relief valve element located in the opening. In one embodiment, the connector is an elbow connector having a tube fitting and a male (Luer) conical lock fitting. The connector is particularly well suited for connecting fluid supply lines with medical devices where the pressure level in the fluid supply line may need to be reduced due to an overpressurization condition.

### FIELD OF THE INVENTION

The present invention relates generally to tube connectors, and more particularly to a tube connector having an integrated relief valve.

### BACKGROUND OF THE INVENTION

A combination of tubing and tube connectors are used for conveying fluids (i.e., gas or liquids) in a wide range of applications. One such application is a surgical fluid management system wherein a combination of tubing and tube connectors serve as a fluid conduit for delivering and/or removing liquid to/from a surgical site in connection with a medical procedure (e.g., irrigation, distention and suction). In some cases, the tubing and tube connectors are fluidly connected with a medical device (e.g., a surgical tool), such as an endoscopic surgical tool that allows a surgeon to conveniently direct liquid to the surgical site and view the surgical site through an optical element. In order to facilitate convenient orientation of the surgical tool during use, it is desirable to use a connector having a rotatable fitting interface to connect the tubing with the surgical tool. In this regard, the rotatable fitting interface allows the surgical tool to be freely moved relative to the tubing. Moreover, the angular bend of the connector allows the tubing to be directed away from the surgical site, thereby allowing unimpeded access thereto.

If delivery of liquid to the surgical site (e.g., body cavity) results in an over-pressurization condition, then a patient may be harmed. Accordingly, the control system of a typical surgical fluid management system is programmed to respond to the over-pressurization condition by reducing or stopping fluid flow to the fluid conduit. However, in the event that the control system fails to respond properly to the over-pressurization condition, one or more pressure relief valves may be disposed in the fluid conduit to discharge liquid, and thereby provide a redundant safety mechanism.

It has been observed that incorporating a pressure relief valve into a fluid conduit requires adding one or more tube connectors to the existing set of tube connectors. This need to increase the number of tube connectors raises the costs associated with use of a surgical fluid management system. Moreover, since all tubing and tube connectors used with the medical device are typically discarded after use in order to maintain sterility, the need for additional tube connectors results in increased waste.

The present invention addresses the drawbacks of the prior art by providing a tube connector having an integrated pressure relief valve.

### SUMMARY OF THE INVENTION

In accordance with the present invention, there is provided a tubing set for conveying a fluid for a medical procedure, the tubing set comprising: at least one tube for delivering the fluid from a fluid source; and a connector having an integrated pressure relief valve element, said connector located at a distal end of the tube for fluidly connecting the tube with a medical device.

In accordance with another aspect of the present invention, there is provided a fluid management system for use with a medical device, said system comprising a first connector for connecting a fluid inlet line to an irrigation port of said medical device. The first connector includes a body having a fluid passageway extending therethrough, wherein a first fitting is formed at a first end of the body and a second fitting is formed at a second end of the body; an opening formed in the body that is in communication with the fluid passageway; and a valve element located in the opening.

In accordance with yet another aspect of the present invention, there is provided a connector comprising: a body having a fluid passageway extending therethrough, wherein a first fitting is formed at a first end of the body and a second fitting is formed at a second end of the body; an opening formed in the body that is in fluid communication with the fluid passageway; and a valve element located in the opening.

An advantage of the present invention is the provision of a tube connector having an integrated relief valve;

Another advantage of the present invention is the provision of a tube connector having an integrated relief valve that facilitates freedom of movement of a device connected thereto;

A still further advantage of the present invention is the provision of a tube connector having an integrated relief valve that directs tubing away from a surgical site;

A still further advantage of the present invention is the provision of a tube connector having an integrated relief valve, thereby minimizing the number of components needed to connect a tube set with a medical device where a pressure relief valve is needed;

Still another advantage of the present invention is the provision of a tube connector having an integrated relief valve, wherein a desired relief valve opening pressure can be easily achieved by simple modifications in the manufacturing process.

Yet another advantage of the present invention is the provision of a tubing set for conveying a fluid, wherein the tubing set includes a tube and a connector having an integrated relief valve.

These and other advantages will become apparent from the following description taken together with the accompanying drawings and the appended claims.

### BRIEF DESCRIPTION OF THE DRAWINGS

The invention may take physical form in certain parts and arrangement of parts, an embodiment of which will be described in detail in the specification and illustrated in the accompanying drawings which form a part hereof, and wherein:

FIG. 1 is a perspective view of an elbow connector with an integrated relief valve, according to a first embodiment of the present invention;

FIG. 2 is a cross-sectional view of the elbow connector shown in FIG. 1, taken along lines 2-2;

FIG. 3 is an exploded partial cross-sectional view of the elbow connector shown in FIG. 1;

FIG. 4 is a perspective view of the relief valve (umbrella check valve) of the elbow connector shown in FIGS. 1-3;

FIG. 5 is a perspective view of an elbow connector with an integrated relief valve, according to a second embodiment of the present invention;

FIG. 6 is a perspective view of an elbow connector with an integrated relief valve, according to a third embodiment of the present invention;

FIG. 7 is a perspective view of an elbow connector with an integrated relief valve, according to a fourth embodiment of the present invention;

FIG. 8 is a cross-sectional view of the elbow connector shown in FIG. 7, taken along lines 8-8; and

FIG. 9 is a cross-sectional view of an elbow connector of the present invention having an integrated relief valve according to a fifth embodiment of the present invention;

FIG. 10 is a cross-sectional view of an elbow connector without a relief valve, according to an alternative embodiment of the present invention;

FIG. 11 is a schematic of a fluid management system connected with an endoscopic surgical tool, wherein the fluid management system comprises a tubing set including a connector having an integrated relief valve according to an embodiment of the present invention;

FIG. 12 is a perspective view of an elbow connector with multiple integrated relief valves, according to a sixth embodiment of the present invention;

FIG. 13 is a cross-sectional view of the elbow connector shown in FIG. 12, taken along lines 13-13;

FIG. 14 is a cross-sectional view taken along lines 14-14 of FIG. 13, wherein a rotatable pin is located in a first position enabling one of the two integrated relief valves; and

FIG. 15 is a cross-sectional view taken along lines 14-14 of FIG. 13, wherein the rotatable pin is located in a second position disabling the two integrated relief valves.

### DETAILED DESCRIPTION OF THE INVENTION

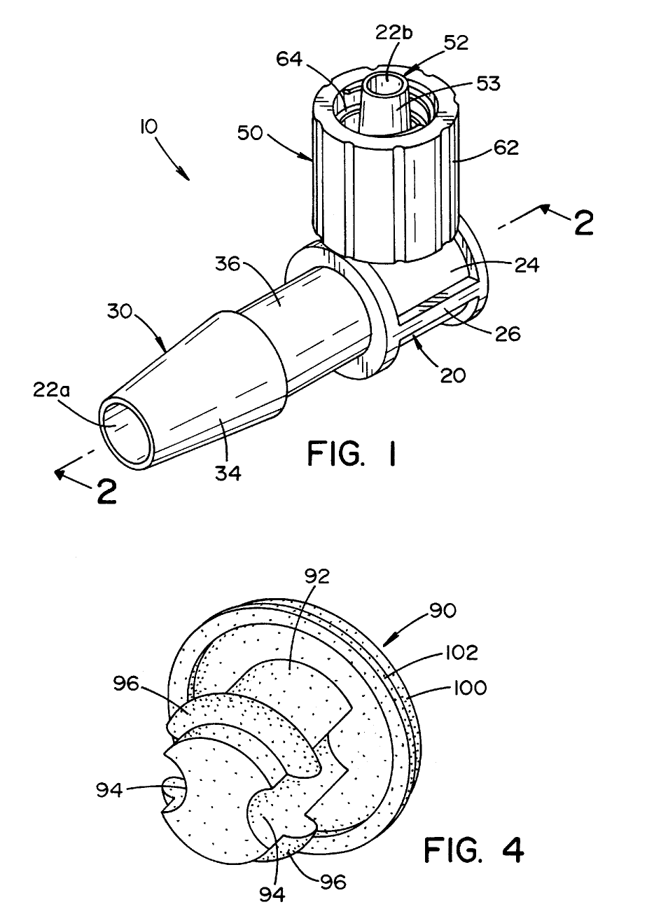

Referring now to the drawings wherein the showings are for the purposes of illustrating embodiments of the invention only and not for the purposes of limiting same, FIGS. 1-3 show a connector 10 according to a first embodiment of the present invention. In this embodiment connector 10 takes the form of an elbow connector. An elbow connector connects the ends of tubes to each other at an angle (i.e., an angular bend) to change the direction of fluid flow. The angle of connection may vary depending upon the application. However, most common elbow connectors connect the ends of tubes at angles of 45 or 90 degrees.

Connector 10 includes a body 20 with a fluid passageway 22 extending therethrough. A first fitting 30 is formed at a first end of body 20 and a second fitting 50 is formed at a second end of body 20. A port or opening 70 formed in body 20 is in communication with fluid passageway 22. In the embodiment shown in FIGS. 1-3, opening 70 includes a circular outer recess 72 and a circular inner recess 74. Circular inner recess 74 defines an annular wall 82 having a thickness (T) that is determined by depth (D) of outer recess 72. Connector 10 also includes a valve element 90 disposed in opening 70. Recessed portions 24 defining ribs 26 may be formed in body 20. The formation of recessed portions 24 reduces material costs for manufacture of connector 10.

Body 20 may be made of a wide variety of suitable materials, including, but not limited to, polycarbonate, polyvinyl chloride (PVC), polyethylene, polypropylene, nylon, polyvinylidene fluoride (PVDF), perfluoroalkoxy (PFA), polytetrafluoroethylene (PTFE), an ABS (acrylonitrile butadiene styrene) polymer, polysulfone, polyether sulfone, polyurethane, polyetherimide and polyetheretherketone. Body 20 may be manufactured by injection molding, or other techniques well known in the art.

In the illustrated embodiment, first fitting 30 is a hose fitting adapted to receive a cylindrical hose. First fitting 30 includes a conical section 34 and a cylindrical section 36. Section 22a of fluid passageway 22 extends through first fitting 30.

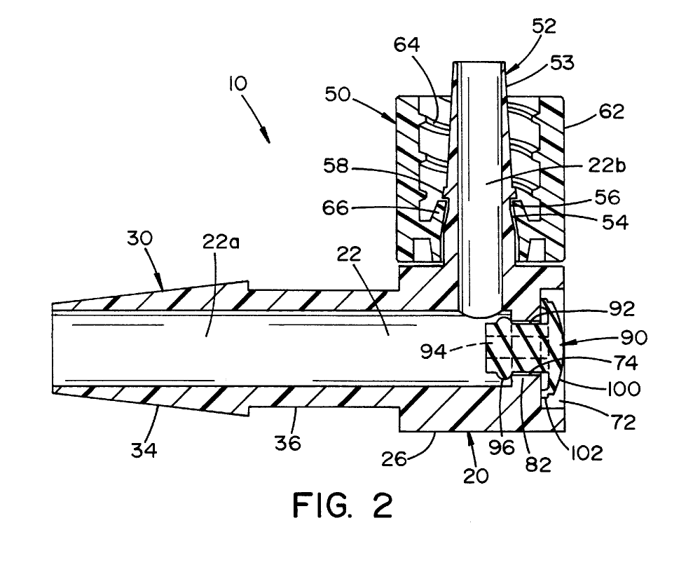

Second fitting 50, according to the illustrated embodiment, is a standard male (Luer) conical lock fitting adapted to receive a standard female (Luer) conical lock fitting (not shown) having an external thread. Second fitting 50 is comprised of a male slip 52 and a rotatable internally threaded collar 62. As best seen in FIG. 3, male slip 52 includes a tapered section 53, a conical section 54, a cylindrical section 56 and an annular flange 58. Collar 62 includes a threaded inner surface 64 and an inner split ring 66 comprised of a plurality of fingers (best seen in FIGS. 2 and 3). Section 22b of fluid passageway 22 extends through second fitting 50.

Collar 62 is mounted onto slip 52 in a snap lock fashion, as conventionally known. In this regard, annular flange 58 of slip 52 captures and retains the fingers of split ring 66, as seen in FIG. 2. Once mounted onto slip 52, collar 62 is rotatable thereabout. The diameter of conical section 54 and/or cylindrical section 56 of slip 52 may be decreased in order to provide an increase in the freedom of movement of collar 62, thereby facilitating alignment and connection of the (Luer) conical lock fitting with a female (Luer) conical lock fitting.

Depending upon the application, it is contemplated that the standard male (Luer) conical lock fitting (second fitting 50) and standard female (Luer) conical lock fitting will comply with European Standard EN 1707 (Conical Fittings with a 6% (Luer) Taper for Syringes, Needles and Certain Other Medical Equipment—Lock Fittings).

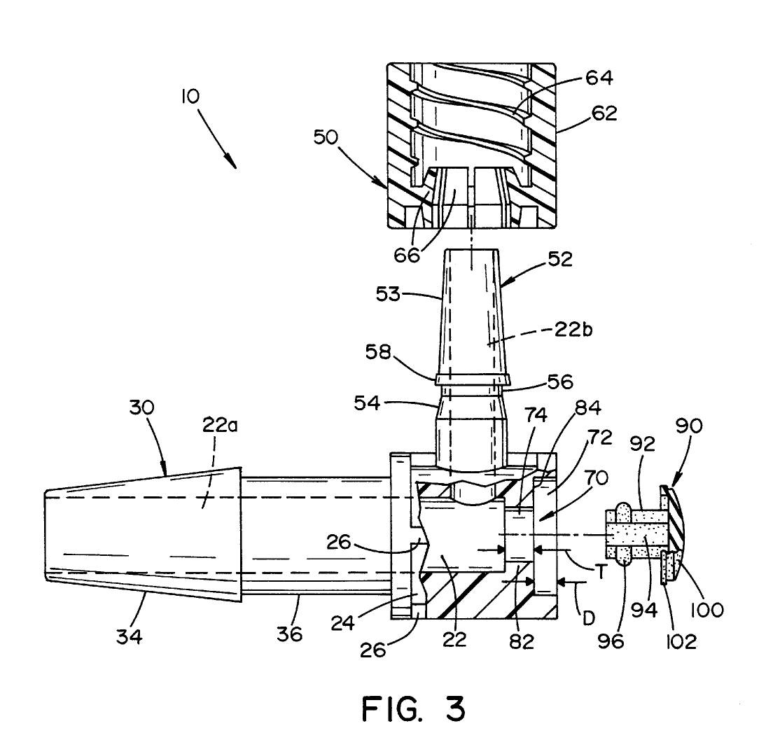

In the first embodiment of the present invention, valve element 90 takes the form of an umbrella check valve that serves as a pressure relief valve, as shown in detail in FIG. 4. The umbrella check valve includes an elongated vented stem 92 having a generally cylindrical configuration, and a flexible annular dome portion 100 having an annular lip 102. Elongated vented stem 92 includes elongated recesses 94 formed therein and flange portions 96. The check valve is preferably made of an elastomeric material, including, but not limited to, silicone, fluorosilicone, nitrile, and EPDM (ethylene propylene diene monomer) rubber.

As shown in FIG. 3, outer surface 84 of annular wall 82 provides a substantially planar surface that serves as a seating surface for valve element 90. In this regard, vented stem 92 is inserted through inner recess 74 such that annular flange 82 is captured between flange portions 96 and dome portion 100 of the umbrella check valve, as best seen in FIG. 2. Dome portion 100 is seated against outer surface 84. The umbrella check valve is normally closed, and opens when a relief pressure is reached. The thickness (T) of annular wall 82 determines the relief pressure of the umbrella check valve. Therefore, the depth (D) of outer recess 72 may be varied (e.g., by mold design or machining) in order to obtain a thickness (T) corresponding to a desired relief pressure.

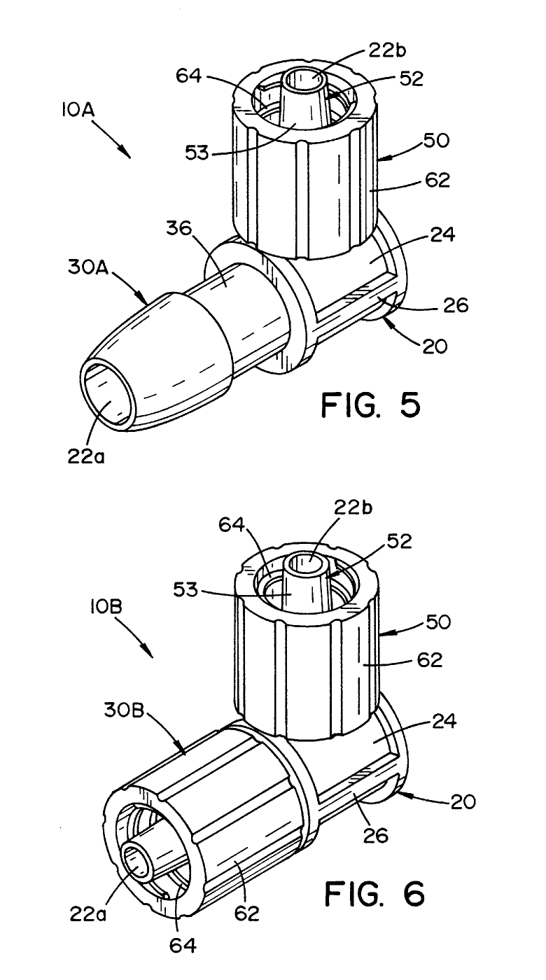

Alternative embodiments of a connector according to the present invention will now be described with reference to FIGS. 5-10 and 12-15. In the drawings of each alternative embodiment, components that are similar to those of connector 10 (described above) bear the same reference numbers.

FIG. 5 illustrates a connector 10A according to a second embodiment of the present invention. Connector 10A is substantially the same as connector 10 described above, except first fitting 30A takes the form of a conventional glue joint rather than a hose fitting.

FIG. 6 shows a connector 10B according to a third embodiment of the present invention. Connector 10B is substantially the same as connector 10 described above, except first fitting 30A takes the form of a standard male (Luer) conical lock fitting adapted to receive a standard female (Luer) conical lock fitting.

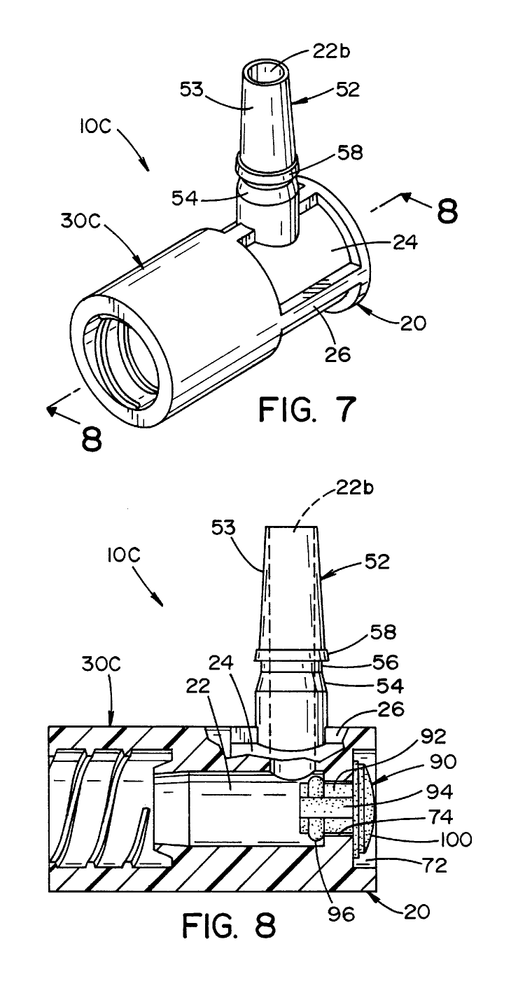

Referring now to FIGS. 7 and 8, these figures illustrate a connector 10C according to a fourth embodiment of the present invention. Connector 10C is substantially the same as connector 10 described above, except first fitting 30C takes the form of a threaded boss fitting. Rotatable internally threaded collar 62 has been omitted from FIGS. 7 and 8 for improved clarity.

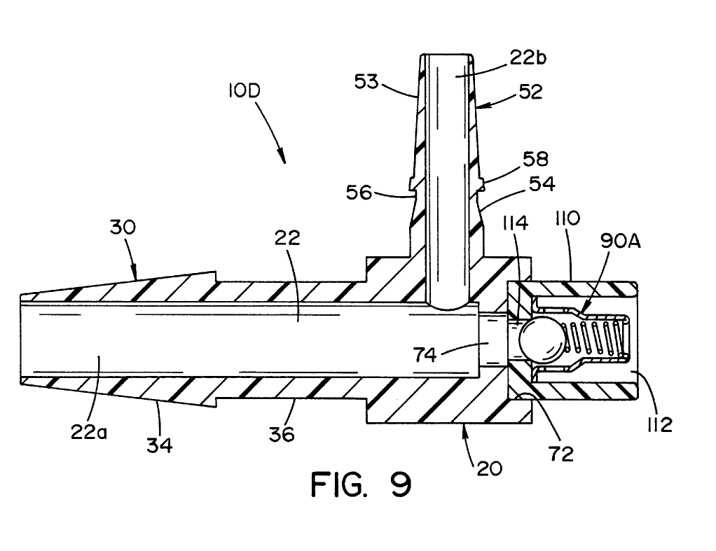

FIG. 9 shows a connector 10D according to a fifth embodiment of the present invention. Connector 10D is substantially the same as connector 10. However, valve element 90A takes the form of a conventional poppet valve. Body 20 includes a generally cylindrical valve housing 110 that defines a recess 112 dimensioned to receive the poppet valve. Housing 110 is dimensioned to be partially received and fixed into outer recess 72. An opening 114 is provided in valve housing 110 and is generally coaxial with inner recess 74. The poppet valve is press fit into recess 112 of valve housing 110. Rotatable internally threaded collar 62 has been omitted from FIG. 9 for improved clarity.

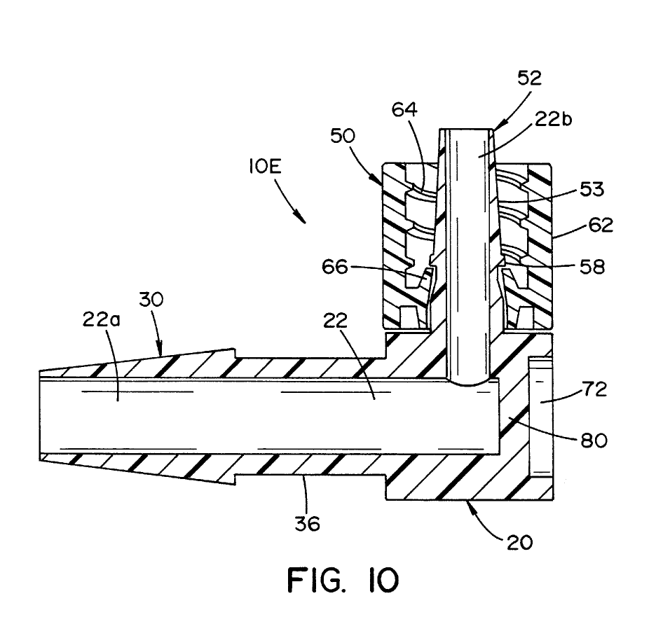

Referring now to FIG. 10, there is shown a connector 10E according to an alternative embodiment of the present invention. In this embodiment, integrated valve element 90 is omitted. Connector 10E may be manufactured in substantially the same way as connector 10 described above. However, inner recess 74 of opening 70 is omitted, thereby providing a closed wall 80. It should be appreciated that in some applications (as will be described below) elbow connectors with and without an integrated relief valve are desired. Connectors 10 and 10E may both be produced by modifications to the same basic manufacturing process.

A connector according to the present invention may also be modified to have a body that includes a plurality of openings 70 adapted to receive either a sealing plug (not shown) for sealing the opening or a valve element serving as a pressure relief valve. In one embodiment, only one opening 70 receives a valve element, while the remaining openings 70 receive a plug. Each opening 70 may be adapted such that the valve element associated therewith responds at a different pressure level (i.e., has a different relief pressure). In this regard, thickness (T) may be varied for each opening 70. Accordingly, a single connector body may be adapted to receive a valve element that provides a different pressure level response depending upon the selected opening 70 receiving the valve element. Alternatively, more than one of the plurality of openings 70 may receive a valve element, where each valve element responds at the same pressure level in order to provide redundant valving, or where each valve element responds at a different pressure level.

Referring now to FIG. 12-15, there is shown a connector 10F according to a sixth embodiment of the present invention. It should be noted that rotatable internally threaded collar 62 has been omitted from FIGS. 12-15 for improved clarity. Connector body 20F includes a generally cylindrical valve housing 210 having a generally cylindrical recess 212 extending therethrough. Recess 212 is coaxially aligned with section 22a of fluid passageway 22.

First and second openings 214 and 216, formed in valve housing 210, are dimensioned to receive a respective valve element 90. In the illustrated embodiment, valve element 90 takes the form of an umbrella check valve, as described in detail above. Valve housing 210 includes raised seating portions 224 and 226 that may have different thicknesses. As a result, valve element 90 located in opening 214 responds at a different pressure level (i.e., has a different pressure relief) than valve element 90 located in opening 216.

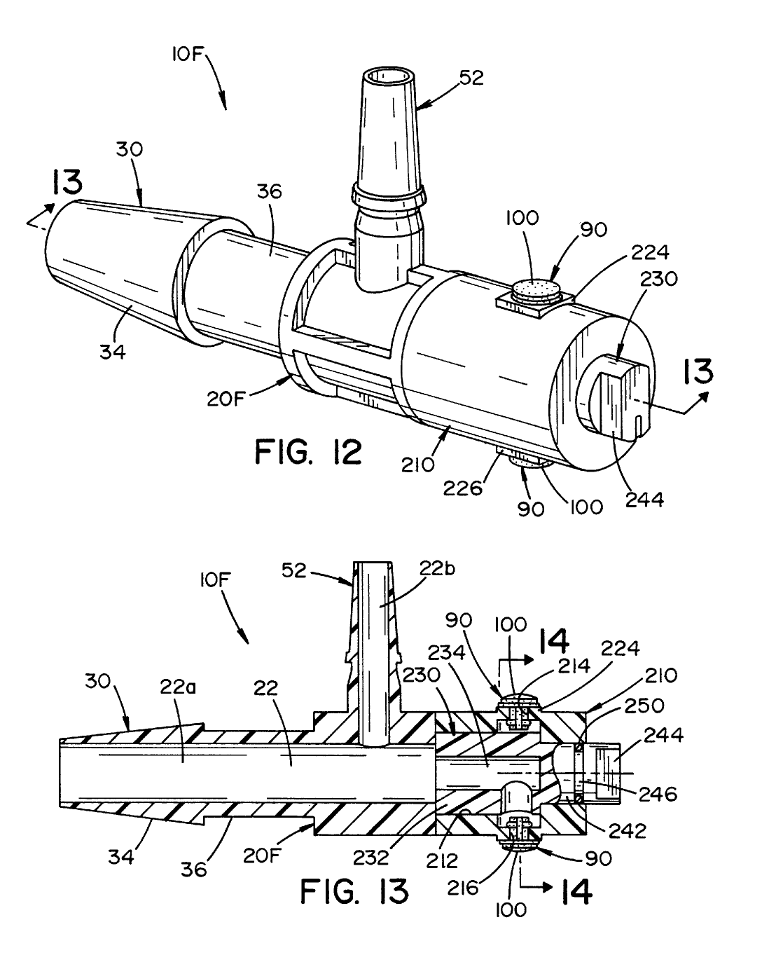

A generally cylindrical rotatable toggle or pin 230 is dimensioned to be captured inside recess 212 of valve housing 210, as best seen in FIG. 13. Pin 230 includes a first portion 232 and a second portion 242. First portion 232 has a generally L-shaped inner recess 234 having one end that is in fluid communication with fluid passageway 22. Second portion 242 includes a handle section 244 that extends outward from valve housing 210. An annular groove 246, formed in second portion 242, is dimensioned to receive an O-ring seal 250.

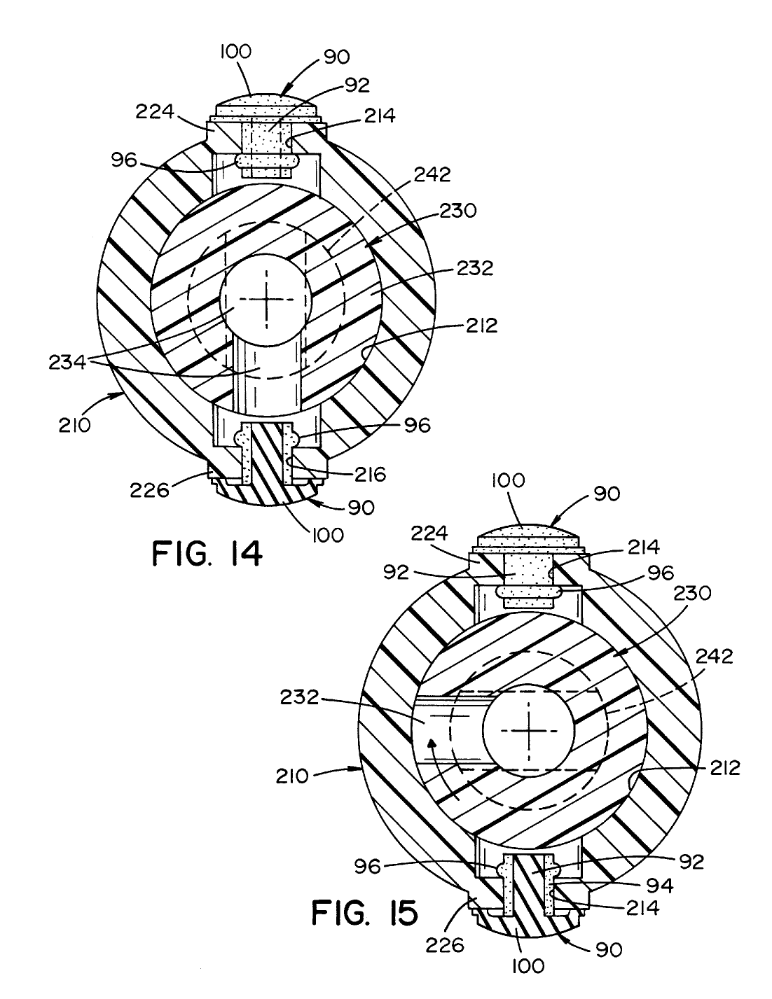

Rotatable pin 230 serves as a selector switch for selecting enablement of one of the two valve elements 90 by aligning inner recess 234 with associated opening 214 or 216 (FIG. 14), or selecting disablement of both valve elements 90 (FIG. 15). Handle 244 allows a user to conveniently rotate pin 230 relative to valve housing 210 to select between the enabled and disabled positions.

According to an alternative embodiment of connector 10F, inner recess 234 of rotatable pin 230 is modified to a T-shape to allow simultaneous enablement of both valve elements 90.

It is contemplated that the connector of the present invention may be adapted to incorporate a variety of different types of relief valves, including, but not limited to, umbrella valves, poppet valves, duckbill valves, flapper valves, needle valves, Belleville valves, dome valves, diaphragm valves, rupture discs and combinations thereof. It is further contemplated that connector 10 may be modified to provide any desired combination of fittings known in the prior art. Moreover, while the illustrated embodiments of the present invention show connector 10 as an elbow connector, it is contemplated that connector 10 may take other desired forms, including, but not limited to Y-connectors, T-connectors, parallel tube connectors, and straight connectors.

It should be appreciated that the illustrated fittings and combinations of fittings are for illustration purposes only and are not to be construed as limiting the present invention. In this regard, it is contemplated that the type of fittings used in connection with the present invention may include, but are not limited to: hose fitting, glue fitting, standard male (Luer) conical lock fitting, female (Luer) conical lock fitting, barbed fitting, stepped fitting, threaded fitting, and proprietary or industry-standard quick disconnects, such as cam and groove type disconnects.

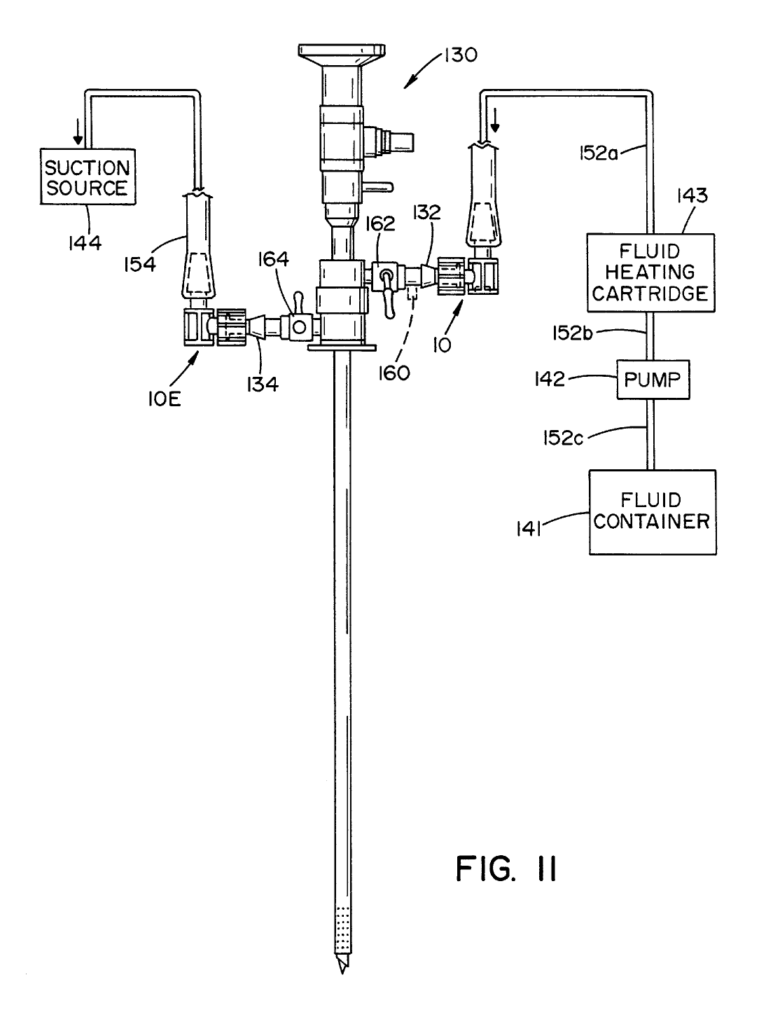

FIG. 11 illustrates connectors of the present invention as components of a surgical fluid management system used in connection with a medical device for a medical procedure. In FIG. 11, the medical device takes the form of a conventional endoscopic surgical tool 130 having an irrigation port 132 and a suction port 134, Irrigation port 132 is in fluid communication with a fluid source or container 141 (containing a fluid, such as a sterile medical liquid) via connector 10 and irrigation tube sections 152a, 152b and 152c that define a fluid inlet line. Connector 10 is located at a distal end of tube section 152a to fluidly connect tube section 152a with irrigation port 132 of surgical tool 130. A pump 142 and a fluid heating cartridge 143 are disposed in the fluid inlet line. Fluid heating cartridge 143 is located downstream of fluid container 141 and pump 142 to heat the fluid prior to entering irrigation port 132. Suction port 134 is in fluid communication with a suction source 144 via a connector 10E and a suction tube 154 that define a suction line. In the illustrated embodiment connector 10; irrigation tube sections 152a and 152b; fluid heating cartridge 143; connector 10E; and suction tube 154 comprise a tubing set. It should be appreciated that a tubing set may be comprised of only tubes and connectors.

Surgical tool 130 includes fluid control valves 162 and 164 for controlling fluid flow at the surgical site. Valve 162 controls the flow of fluid being delivered to the surgical site, while valve 164 controls the flow of fluid being removed from the surgical site by suction source 144. Fluid container 141, pump 142, fluid heating cartridge 143 and suction source 144 are components of the fluid management system. Fluid management system is not shown in its entirety, and includes pressure and flow sensors (not shown) and control means for controlling operation of pump 142, suction source 144, and a heater (not shown) for warming fluid in cartridge 143. For example, the fluid management system may take the form of the fluid management system disclosed in U.S. patent application Ser. No. 12/720,475, fully incorporated herein by reference.

In the illustrated embodiment, connectors 10 and 10E take the form of 90-degree elbow connectors that facilitate convenient orientation of endoscopic surgical tool 130 during use. In this regard, the rotatable standard male (Luer) conical lock fitting allows endoscopic surgical tool 130 to move freely relative to the tubing. Moreover, the angular bend of the 90-degree elbow connectors permits the tubing to be directed away from the surgical site, thereby allowing unimpeded access thereto.

In the event that the pressure level exceeds a predetermined pressure threshold limit, the control means of the surgical fluid management system will ordinarily take action to reduce the pressure level (e.g., reduce pump motor speed). However, if the control means fails to take appropriate action, then valve element 90 (i.e., the pressure relief valve) will respond by opening to allow the release of fluid, thereby reducing the pressure level. The predetermined pressure threshold limit may vary depending upon the type of medical procedure (e.g., 30 to 300 mmHg).

It is also contemplated that a pressure relief valve 160 may be integrated directly into surgical tool 130, thereby eliminating the need for integrated valve element 90 of connector 10.

The foregoing description is a specific embodiment of the present invention. It should be appreciated that this embodiment is described for purposes of illustration only, and that numerous alterations and modifications may be practiced by those skilled in the art without departing from the spirit and scope of the invention. It is intended that all such modifications and alterations be included insofar as they come within the scope of the invention as claimed or the equivalents thereof.

Having described the invention, the following is claimed:

1. A tubing set for conveying a fluid for a medical procedure, the tubing set comprising:
at least one tube for delivering the fluid from a fluid source; and
a connector having an integrated pressure relief valve element, said connector located at a distal end of the tube for fluidly connecting the tube with a medical device.

2. A tubing set according to claim 1, wherein said connector comprises:
a body having a fluid passageway extending therethrough, wherein a first fitting is formed at a first end of the body and a second fitting is formed at a second end of the body;
an opening formed in the body that is in communication with the fluid passageway, wherein said valve element is located in the opening.

3. A tubing set according to claim 2, wherein said opening includes an outer recess and an inner recess, wherein the inner recess defines a wall having a thickness (T) that is determined by depth (D) of the outer recess.

4. A tubing set according to claim 2, wherein said first fitting is a hose fitting adapted to receive a hose and said second fitting is a male (Luer) conical lock fitting adapted to receive a female (Luer) conical lock fitting.

5. A tubing set according to claim 2, wherein said first and second fittings are selected from the group consisting of the following: hose fitting, glue fitting, standard male (Luer) conical lock fitting, female (Luer) conical lock fitting, barbed fitting, stepped fitting, threaded fitting proprietary quick disconnects and industry-standard quick disconnects.

6. A tubing set according to claim 2, wherein said valve element is an umbrella valve having an elongated vented stem and a flexible dome portion.

7. A tubing set according to claim 2, wherein said valve element is selected from the group consisting of the following: umbrella valve, poppet valve, duckbill valve, flapper valve, needle valve, Belleville valve, dome valve, diaphragm valve, rupture discs and combinations thereof.

8. A tubing set according to claim 2, wherein said connector is an elbow connector.

9. A tubing set according to claim 8, wherein said first and second fittings are at an angle of 45 or 90 degrees relative to each other.

10. A fluid management system for use with a medical device, said system comprising:
a first connector for connecting a fluid inlet line to an irrigation port of said medical device, said first connector comprising:
a body having a fluid passageway extending therethrough, wherein a first fitting is formed at a first end of the body and a second fitting is formed at a second end of the body;
an opening formed in the body that is in communication with the fluid passageway; and
a valve element located in the opening.

11. A fluid management system according to claim 10, wherein said system further comprises a pump for supplying a fluid to the fluid inlet line from a fluid container.

12. A fluid management system according to claim 11, wherein said system further comprises a fluid heating cartridge disposed in said fluid inlet line.

13. A fluid management system according to claim 10, wherein said first connector is an elbow connector.

14. A fluid management system according to claim 10, wherein said first and second fittings are at an angle of 45 or 90 degrees relative to each other.

15. A fluid management system according to claim 10, wherein said fluid management system further comprises:
a second connector for connecting a suction line to a suction port of said medical device, said second connector comprising a body having a fluid passageway extending therethrough, wherein a first fitting is formed at a first end of the body and a second fitting is formed at a second end of the body.

16. A connector comprising:
a body having a fluid passageway extending therethrough, wherein a first fitting is formed at a first end of the body and a second fitting is formed at a second end of the body;
an opening formed in the body that is in fluid communication with the fluid passageway; and
a valve element located in the opening.

17. A connector according to claim 16, wherein said opening includes an outer recess and an inner recess, wherein the inner recess defines a wall having a thickness (T) that is determined by depth (D) of the outer recess.

18. A connector according to claim 16, wherein said first fitting is a hose fitting adapted to receive a hose and said second fitting is a male (Luer) conical lock fitting adapted to receive a female (Luer) conical lock fitting.

19. A connector according to claim 16, wherein said first and second fittings are selected from the group consisting of the following: hose fitting, glue fitting, standard male (Luer) conical lock fitting, female (Luer) conical lock fitting, barbed fitting, stepped fitting and threaded fitting.

20. A connector according to claim 16, wherein said valve element is an umbrella check valve having an elongated vented stem and a flexible dome portion.

21. A connector according to claim 16, wherein said body includes a plurality of openings, wherein said valve element is located in one of said openings and a sealing plug is located in each of the other said openings.

22. A connector according to claim 21, wherein said valve element provides a different pressure level response depending upon the opening selected to receive the valve element.

23. A connector according to claim 16, wherein said body includes a plurality of openings that are in fluid communication with the fluid passageway, and a plurality of valve elements respectively located in each of said plurality of openings.

24. A connector according to claim 23, wherein said connector further comprises a selector switch for selecting enablement of one or more of said plurality of valve elements.
